(19) United States
(12) Patent Application Publication
(10) Pub. No.: US 2024/0409874 A1
(43) Pub. Date: Dec. 12, 2024

(54) MULTI-CONDUIT, MICROELECTRODE ARRAY (MEA) DEVICE HAVING MICROCHAMBER FOR ELECTROPHYSIOLOGICAL STUDIES

(71) Applicant: UNIVERSITY OF CENTRAL FLORIDA RESEARCH FOUNDATION, INC., Orlando, FL (US)

(72) Inventors: Swaminathan RAJARAMAN, Winter Park, FL (US); Jorge MANRIQUE CASTRO, Orlando, FL (US)

(21) Appl. No.: 18/677,956

(22) Filed: May 30, 2024

Related U.S. Application Data
(60) Provisional application No. 63/506,840, filed on Jun. 8, 2023.

(51) Int. Cl. C12M 1/34; C12M 3/00; C12M 3/06; G01N 33/487

(52) U.S. Cl. CPC C12M 41/46; C12M 21/08; C12M 23/16; G01N 33/48707

(57) ABSTRACT

A monolithically 3D printed array of microchannels in a multilayer circuit includes a base substrate and integrated microchamber to form a microelectrode array (MEA) device. Microchannels at different levels serve as conduits towards a centrally located 2.5D/3D Microelectrode Array (MEA) for electrical stimulation/recording of electrogenic spheroids, and as inlet/outlet for injection/suction of liquids, e.g., samples or reagents. The microchamber allows for control and isolation of the cultured microenvironment, and additionally perfusion of gases, such as O₂ and CO₂ for electroactive responses. The device is operative with organoids under Phosphate Buffer Saline (PBS) and sample gas (Oxygen) injection.

### PRIORITY APPLICATION(S)

This application claims priority to U.S. provisional Application No. 63/506,840 filed Jun. 8, 2023, the disclosure which is hereby incorporated by reference in its entirety.

### FIELD OF THE INVENTION

The present invention relates to the field of microelectrode arrays and related microfluidics, microconduits, multiphase and similar packaging systems, and more particularly, this invention relates to a three-dimensional (3D) microelectrode array (MEA) device having a microchamber and electrophysiological and multiple sensing capabilities.

### BACKGROUND OF THE INVENTION

There are several assays and tests in a typical medical laboratory that require reliable biomedical platforms in order to provide accurate measurements, and fast responses upon analyte-antigen interaction in applications like drug screening, toxicity studies, and personalized treatments.

Lab-on-a-Chip (LoC) started as an ideal solution for detection and biochemical assays making use of microfluidic platforms monolithically integrated to optical interfaces. Addition of cells and tissue within the next generation of LoC platforms made them ideal to mimic organs and reduce the gap between in-vivo and in-vitro experiments. These platforms known as Organ-on-a-Chip (OoC) serve as drug testing modeling systems and are making rapid strides in replacing the inaccurate predictability emanating from animal testing and 2D cell cultures. Tailoring drug dosages are still a challenge, but even more complex and highly engineered biomedical platforms are being developed to face this challenge by incorporating multiple organs in a single device.

Human-on-a-Chip (HoC) is the latest innovation in LOC systems to evaluate drug toxicity and targeting through pharmacokinetic and pharmacodynamic profiles. On the other hand, multi-size cellular entities named organoids provide the next generation of in-vitro models for human physiology and personalized medicine. Because organoids are 3D dimensional cell constructs, it is important to have a good understanding of their self-assembly processes and innate microenvironments for clinical studies.

Organoids respond differently to external agents depending on the stage of development. For this reason, there is a large interest in the Biological Micro Electro Mechanical Systems (BioMEMS), LoC and bioelectronic device communities for capturing the effect of multiple stimuli on these biomedical entities. However, most state-of-the-art platforms are still lacking in the integration of a controlled microenvironment to sensing/stimulus systems.

Oxygen is a well-known energy source for metabolic processes and cellular development such as embryonic stem cell differentiation, organogenesis, and morphogenesis. As such, new strategies to avoid hypoxic conditions, and oxygenating biomaterials integration can be performed using electrical signal recordings emanating from oxygen transport.

Development of microelectrode array (MEA) devices to study organoids is an area of continuing development and includes flexible MEA devices that envelope spheroids, multi-modal sensors with shape control on assembloids, and multiplexing sensing modalities on the same chip. Intimate interfacing with the biological entity is highly desired. New designs, exploration of novel materials, and pushing traditional microfabrication processes to their limits is being pursued in the microfabrication techniques for these BioMEMS platforms. Further developments are desirable.

### SUMMARY OF THE INVENTION

A three-dimensional (3D), microchannel-based, microelectrode array (MEA) device for electrophysiological studies may comprise a base substrate having a top face, at least one microchamber secured onto the top face, and a first plurality of microconduits formed within the base substrate. Each of the first plurality of microconduits may comprise a first set of ports on the top face outside the at least one microchamber, and a second set of ports on the top face within the at least one microchamber. A metal fills the microconduits to form a microelectrode array (MEA) on the top face within the at least one microchamber and contact pads on the top face outside the at least one microchamber. At least one additional second microconduit includes a first set of fluid ports on the top face outside the microchamber and a second set of fluid ports on the top face within the at least one microchamber.

The base substrate may comprise a 3D printed substrate. The metal may comprise liquid metal, such as Galinstan, Gallium, Eutectic Gallium-Indium (EGaIn) and Mercury. The MEA and contact pads may each comprise a silver-ink cap or material, such as gold, copper, solder, platinum, tin, and/or similar metal, encapsulating the liquid metal within the first plurality of microconduits. The at least one microchamber may include at least one set of and input output microfluidic ports. Organoids may be within the at least one microchamber on the top face at the MEA. The organoids may be biological or synthetic or any 3D biological entity, or other regular organoids or 3D biological constructs, including spheroids, assembloids, and similar constructs. The microelectrode array may comprise microelectrodes having a maximum-minimum height difference about 300 micrometers.

### BRIEF DESCRIPTION OF THE DRAWINGS

Other objects, features and advantages of the present invention will become apparent from the Detailed Description of the invention which follows, when considered in light of the accompanying drawings in which:

FIGS. 1A and 1B are schematic images illustrating a 3D MEA device having a multilevel (L1-L3) configuration with its microfabrication process shown in FIG. 1A, and images for the microfluidic and reaction chamber of the 3D MEA device shown in FIG. 1B in accordance with a non-limiting example of the invention.

FIGS. 2A-2D are plan view images of contact pads showing a Galinstan liquid metal in FIGS. 2A and 2C and Ag-paste metallization in FIGS. 2B and 2D, and cross-sectional views of empty microchannels in FIG. 2E, a partially filled microchannel in FIG. 2F, and a bimetal interface condition in FIG. 2G.

FIG. 3A are microelectrode focused laser confocal microscopy images and an associated graph profile of a microelectrode.

FIG. 3B is a 3D surface profile image of a microelectrode.

FIG. 3C is a roughness area image of a microelectrode.

FIGS. 4A-4E are images showing the synthetic organoid fabrication used with the 3D MEA device, while FIGS. 4F and 4G are images showing the positioning of the organoids within the multi-phase chamber for the 3D MEA device.

FIG. 4H is a schematic diagram showing the three levels of the 3D MEA, where the first two levels are metal and the third level is fluid with a phantom diagram on the left and block diagram on the right.

FIGS. 5A-5D are EIS spectrum graphs (Bode and Nyquist) for two microchamber conditions with PBS shown in FIGS. 5A and 5C, and O₂ enriched organoids shown in FIGS. 5B and 5D.

FIG. 6 is a high-level flowchart of a method of making the 3D MEA device.

### DETAILED DESCRIPTION

Different embodiments will now be described more fully hereinafter with reference to the accompanying drawings, in which preferred embodiments are shown. Many different forms can be set forth and described embodiments should not be construed as limited to the embodiments set forth herein. Rather, these embodiments are provided so that this disclosure will be thorough and complete, and will fully convey the scope to those skilled in the art.

Among microfabrication techniques, 3D printing (additive manufacturing) provides an efficient way to develop rapid prototypes of various microelectrode arrays (MEAs), reducing the gap between research-based and commercially available devices. With the advent of liquid metals for use in flexible electronics, their use in cardiac organoids studies has gained attention such as the work from Kim et al., “Multimodal Characterization of Cardiac Organoids Using Integrations of Pressure-Sensitive Transistor Arrays with Three-Dimensional Liquid Metal Electrodes,” Nano Letters, Vol. 22, No. 19; pages 7892-7901; Sep. 22, 2022; the disclosure which is hereby incorporated by reference in its entirety. This work discloses integrating several layers of sensing modalities at the expense of fabrication complexity.

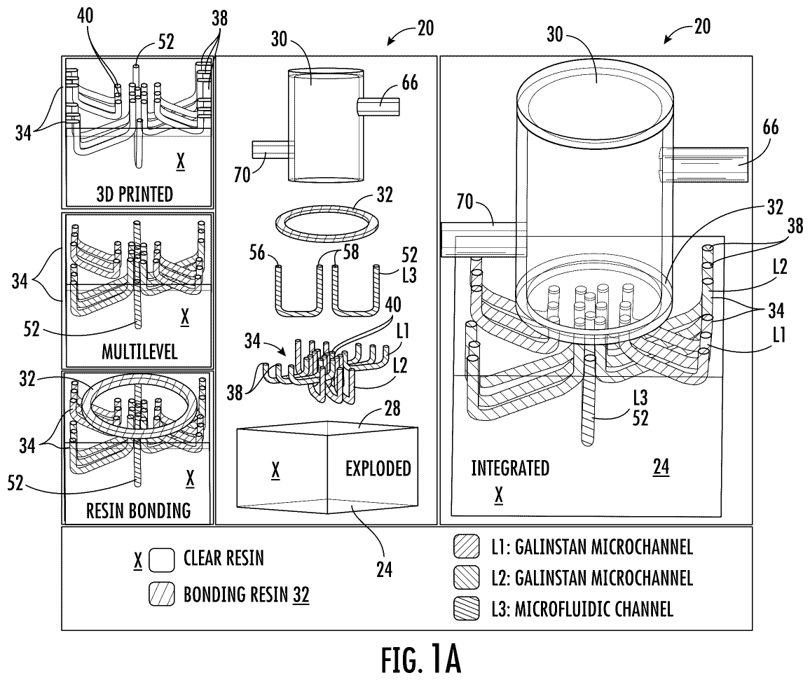

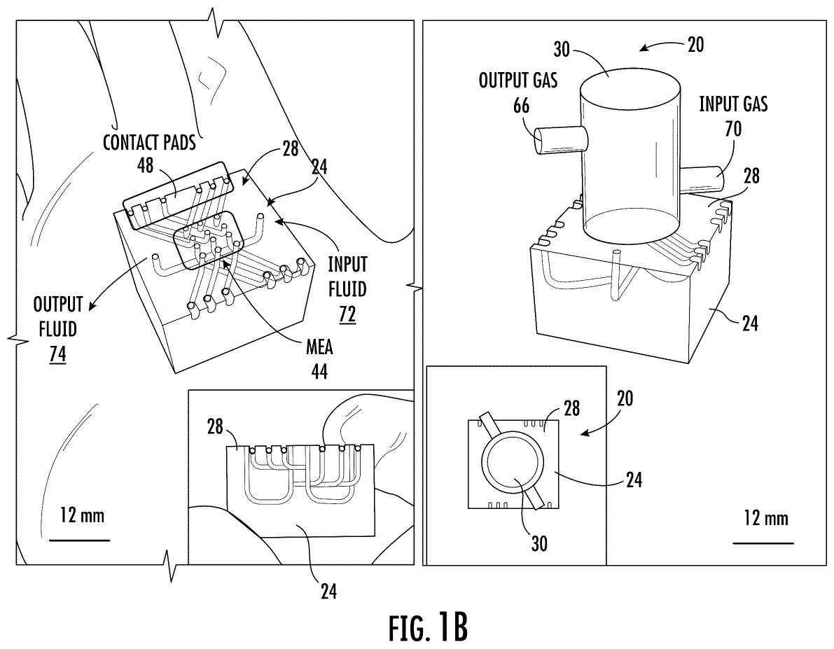

In accordance with a non-limiting example, generally a two-step process is described to produce the 3D printed, packaged, multilevel, microfluidic platform as an MEA device shown at 20 in FIGS. 1A and 1B. This MEA device 20 has an innovative metallization approach using a liquid metal, such as Galinstan, Eutectic Gallium-Indium (EGaIn), and Mercury. It is possible to use other liquid metals. This MEA device 20 includes enhanced multi-modality for liquid and gaseous control in a microchamber microenvironment. In the description that follows, the materials and methods of fabrication of the MEA device 20 are described, followed by a description of the double-metallization for electrode capping, the electrical and physical characteristics, synthetic organoid development and the results.

In accordance with a non-limiting example, the three-dimensional (3D), microchannel-based, microelectrode array (MEA) device 20 for electrophysiological studies is shown schematically at the right hand side of FIG. 1A and an image at the right hand side of FIG. 1B. The MEA device 20 includes a base substrate 24 having a top face 28 and at least one microchamber 30 secured onto the top face. The MEA device 20 may include a plurality of microchambers 30 for high throughput. The at least one microchamber 30 may be resin bonded by a bonding resin 32 and contains microelectrodes 44 and microfluidic ports for gas exchange and flow monitoring. A first plurality of microconduits 34 such as first and second levels (L1 and L2) are formed within the base substrate 24. Each of the first plurality of microconduits 34 may include a first set of ports 38 on the top face 28 outside the microchamber 30 and a second set of ports 40 on the top face 28 within the microchamber 30. A liquid metal 42, such as Galinstan or other metals such as Eutectic Gallium-Indium (EGaIn), Mercury and similar liquid metals, may fill the microconduits 34 to form an array of microelectrodes 44 forming the microelectrode array (MEA) on the top face 28 within the microchamber 30, and contact pads 48 on the top face outside the microchamber 30. The microelectrodes 44 together as an array may also be termed an MEA 44. At least one additional second fluid microconduit 52, and in this example, two second fluid microconduits include a first set of fluid ports 56 on the top face 28 outside the microchamber 30 and a second set of fluid ports 58 on the top face within the microchamber and form a third level (L3) microfluid channel. This at least one additional second fluid microconduit 52 may be formed as a second plurality of fluid microconduits with multiple sets of fluid ports 56, 58.

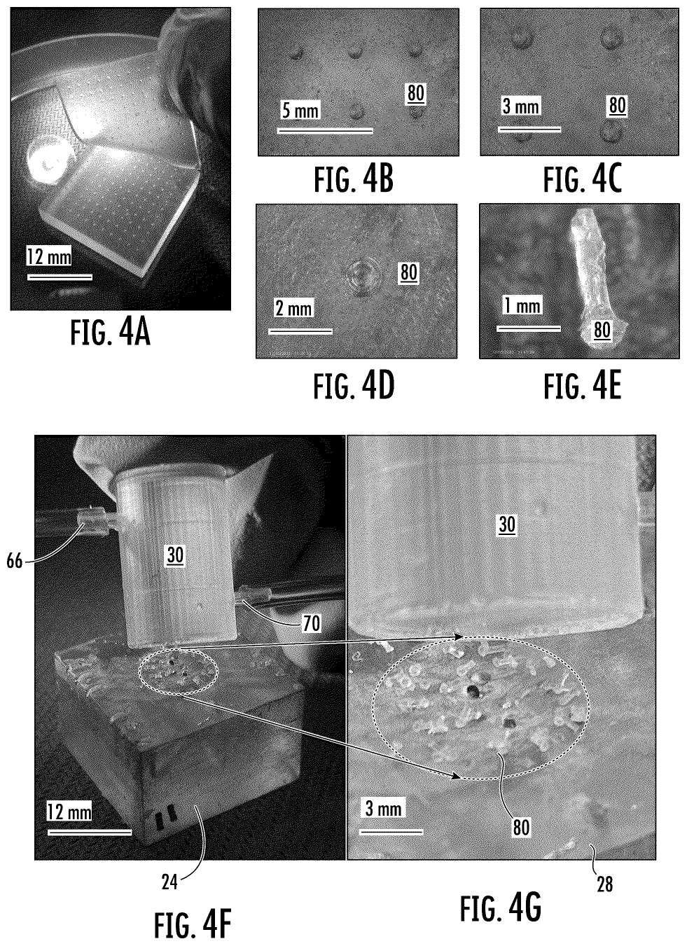

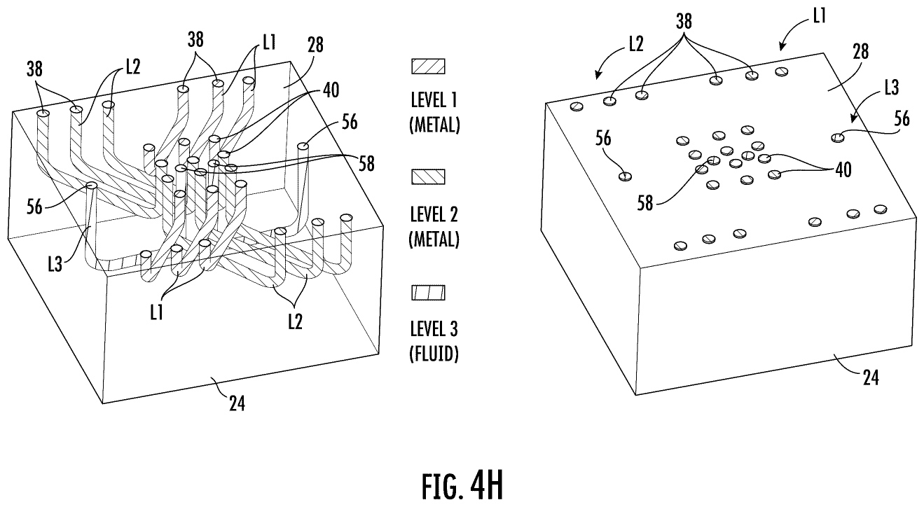

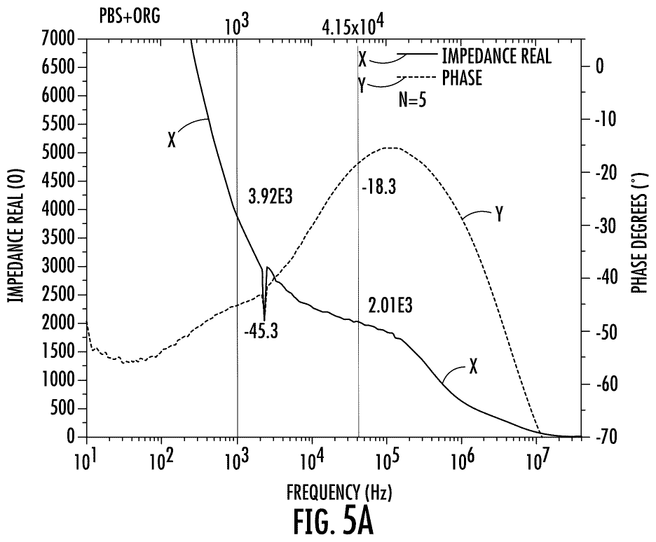

The base substrate 24 may be manufactured as a 3D printed substrate. Different metals 42 may be used, but it has been found that a liquid metal, such as Galinstan, Gallium, Eutectic Gallium-Indium (EGaIn) and Mercury, is acceptable since those metals, e.g., Galinstan as an alloy of Gallium, indium and tin, is liquid at room temperature. The MEA 44 and contact pads 48 may each include a silver-ink cap 62 encapsulating the liquid metal 42 within the first plurality of microconduits 34. The microchamber 30 may include an output fluid such as a gas port 66, and an input fluid such as a gas port 70 as best shown in the right hand image of FIG. 1B. Organoids 80 may be within the microchamber 30 on the top face 28 at the MEA 44 (FIGS. 4F and 4G). The organoids may be biological or synthetic, and in some examples, any 3D biological entity, including 3D biological constructs, spheroids, assemboids, and similar constructs. The microelectrode array may include individual microelectrodes 44 having a maximum-minimum height difference about 300 micrometers so that the MEA has some variation.

MATERIALS AND METHODS: Multilevel Chip Microfabrication. The MEA device 20 that includes the multilevel microfluidic base substrate 24 with its components and reaction microchamber 30 for sample containment were designed using a computer aided design (CAD) program, in this example, Solidworks, with example dimensions of L:24 mm, W:24 mm, H:15 mm. The base substrate 24 and reaction microchamber 30 were micro-stereolithographically 3D printed, such as using Form 3, Formlabs. The MEA 44 was microfabricated by filling 12 microconduits 34 (FIG. 1A) as the first plurality of microconduits (D:1 mm) that were routed at two different levels with liquid metal Galinstan 42 and then metal ink-capped 62, such as by using silver, gold, copper, platinum, solder, tin, and similar materials to encapsulate and generate a 2.5D/3D electrode shape, and in an example, a 3×4 array. The microchamber 30 and multilevel base substrate 24 as a platform were bonded together using a UV-sensitive resin along the edges and cured at 60° C. for 10 mins (FIG. 1A) showing the bonding resin at 82. The multiphase microchamber 30 allows fluid flow of different fluids such as gaseous species through the inlet and outlet ports 70,66 (FIG. 1B) to control the sample microenvironment. Two microfluidic ports (D:1 mm) at a third level (L3) as the at least one fluidic microconduit 58 were left unfilled to serve as input/output ports 72,74 for reagents, sample injection/ejection, washing and flushing operations as shown in FIG. 1B on the left hand view.

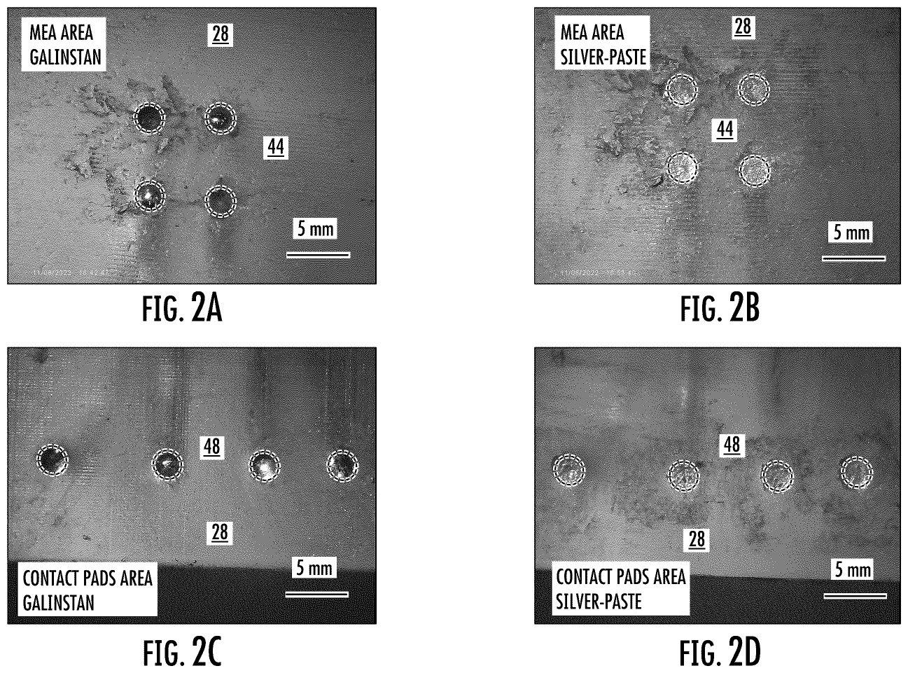

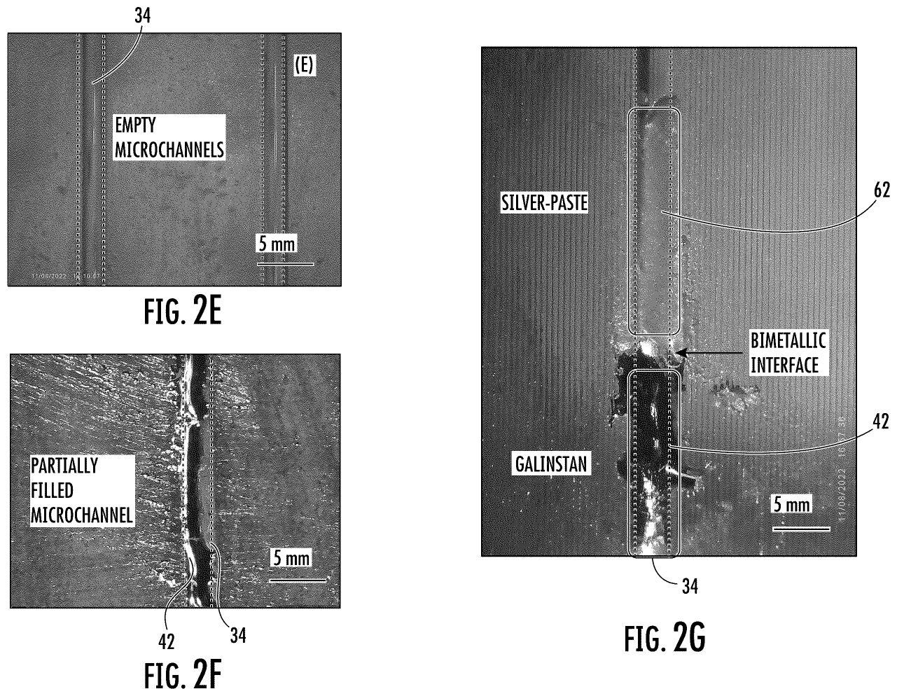

Double-Metallization (Electrode Capping). The first step in the double-metallization process was the filling of liquid metal, such as low melting point (19° C.) Galinstan liquid metal 42 along the microconduits 34 at the first two levels (L1 and L2) shown in FIG. 1A of the base substrate 24 corresponding to the first plurality of microconduits. Other liquid metals may be used, including Gallium, Eutectic Gallium-Indium (EGaIn) or Mercury. In an example, metal injection was performed at room temperature by applying positive pressure on a Galinstan-filled syringe towards the contact pad 48 port corresponding to the outer ports from the microchamber 30 to be secured later, and filling the microconduits 34 in a serial fashion. The capping process with the example silver-ink cap 62 was carried out by adding biocompatible silver paste at the sites corresponding to the microelectrode 44 and areas where contact pads 48 are located, followed by a curing procedure at 100° C. for 24 hours. FIGS. 2A-2D are images showing an array of microelectrodes in FIGS. 1A and 1B and the contact pads in FIGS. 1C and 1D. The silver-ink capping process sealed the microelectrodes 44 and contact pads 48, thus encapsulating the liquid metal 42 inside the first plurality of microconduits 34.

Electrical and Physical Characterization. Conductivity tests were conducted along the metallized microconduits 34. Open and closed channels were tested in order to identify the Galinstan distribution along the microchannel inner surfaces and the junction depth (distance from the Galinstan+Silver alloy to the surface) as shown in FIGS. 2E-2G showing the reference distance of 5 millimeters and two empty microchannels in FIG. 2E, a partially filled microchannel in FIG. 2F, and the bimetallic interface in FIG. 2G with the Galinstan as the metal 42, the silver-ink cap 62 as a silver paste, and between the bimetallic interface. End-to-end electrical resistance measurements of the microelectrode 44 conduits 34 were performed using a multimeter and compared to what could be obtained in theory under the assumption of fully filled Galinstan microconduits 34 and a near-zero junction depth.

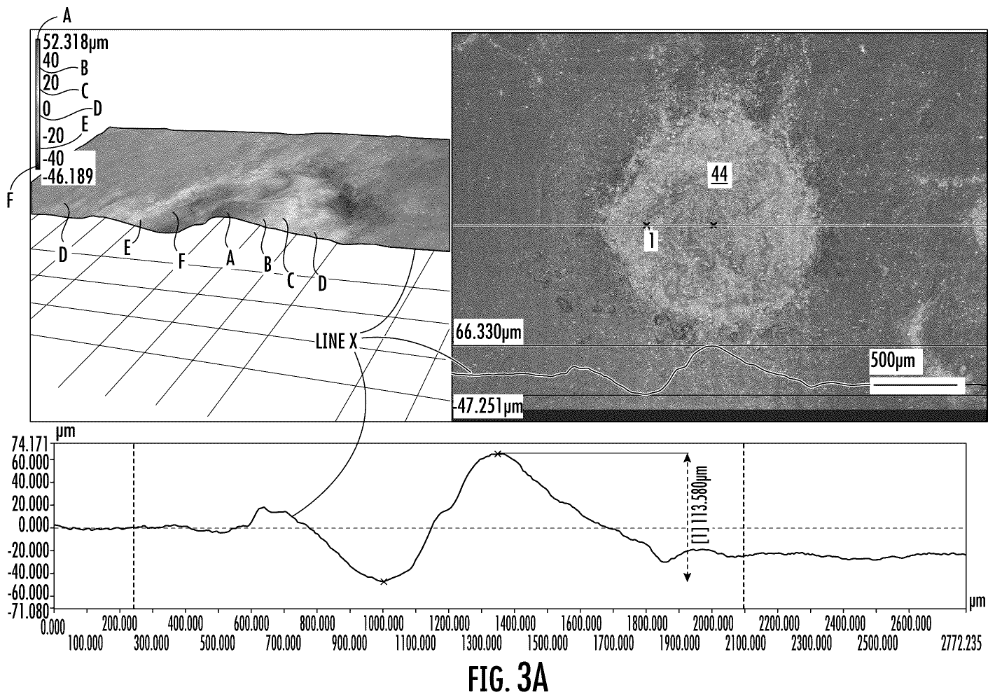

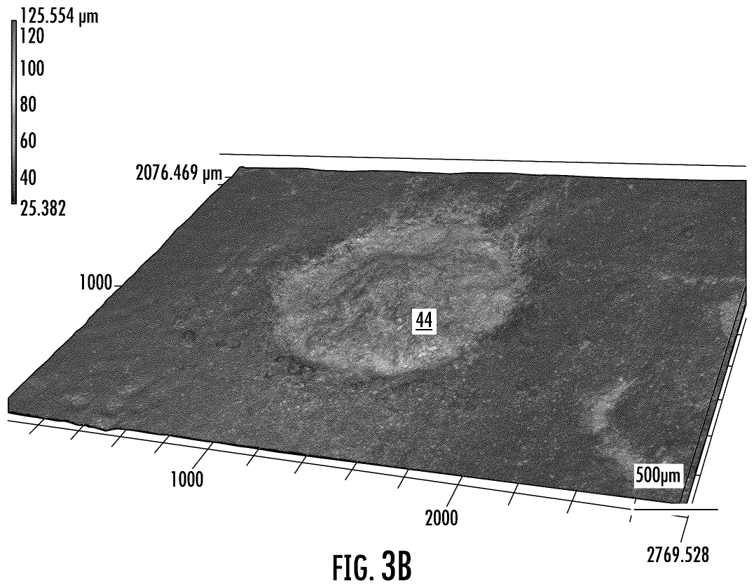

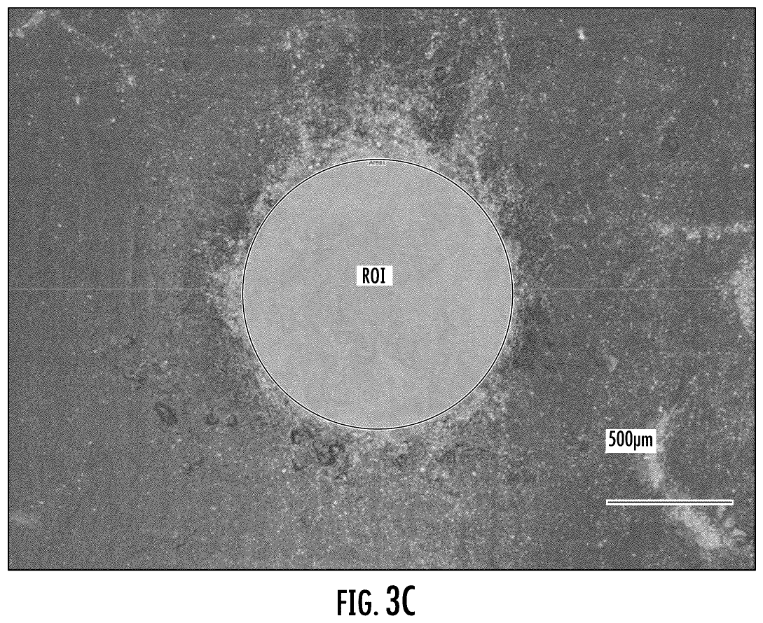

Physical characterization tests that included surface roughness and surface profile measurements for microelectrodes 44 were conducted by laser confocal profilometry. The different profile measurements are shown in FIG. 3A. A 3D surface profile image is shown in FIG. 3B. The topographic mapping is shown in the top left of FIG. 3A, and a plan view image in the top right. The elongated graph at the bottom of FIG. 3A shows the enlarged line X corresponding to lines X in the two top left and right views. The roughness for a microelectrode is shown in the area of evaluation for the microelectrode surface roughness with an average of 56 micrometers in FIG. 3C. The circular geometric area (D:1 mm) of the electrodes 44 was selected as the region of interest (ROI) shown as the solid, shaded circle for both measurements (FIG. 3C). To quantify 2.5D/3D microelectrode features after electrode capping with the silver-ink cap 62, the Max-Min (maximum-minimum) measurement was obtained within the ROI.

Electrochemical Impedance Spectroscopy (EIS) measurements were conducted from 10 Hz to 40 MHz to determine the frequency response (Bode and Nyquist plots) of the organoids 80 under Phosphate Buffer Saline (PBS) environment injected through the microfluidic ports 72, 74, before and after O₂ addition through the microchamber ports as the gas ports 66,70. Barrier integrity and coverage of synthetic organoids 80 onto the microelectrode array 44 were calculated from specific frequency response of the MEA device 20 serving as a platform at 1 kHz and 41.5 kHz respectively. A second level (L2) silver electrode (FIG. 1A) within the MEA 44 served as a counter electrode.

Organoid Development. A 10×10 matrix of synthetic organoids 80 (D:500 μm each) was created with Polydimethylsiloxane (PDMS) (10:1 ratio) and polystyrene (PS) beads (D:10 μm) as shown in FIGS. 4A-4E showing the larger matrix in FIG. 1A with reference at 12 millimeters, and diminishing size references of 5 millimeters (FIG. 4B), 3 millimeters (FIG. 4C), 2 millimeters (FIG. 4D) and 1 millimeter (FIG. 4E). However, as noted before, the organoids may be biological or synthetic or any 3D biological entity. To control gas diffusion through the sample, in an example, organoids 80 mixing was composed of 3 grams of PDMS plus 20 μL polystyrene. Once the matrix was cured, the organoids 80 as synthetic organoids in this example were sectioned using a biopsy punch tool, and placed atop the MEA 44 area prior to chip bonding (FIGS. 4F and 4G) of the microchamber 30 to the base substrate 24. The 12 millimeter reference image is shown in FIG. 4F and the enlarged 3 millimeter reference image is shown in FIG. 4C.

Results. Experimental resistance measurements for the microchannels 34 in the multi-level circuit, levels 1 (L1) and 2 (L2) were 2.2 Ω and 1.4 Ω (Ohms) respectively as shown in the setup in the schematic of FIG. 4H with the left hand view showing the phantom view and the right hand view showing the solid base substrate 24. These values were later compared to the calculated theoretical results of 0.007 Ω and 0.012 Ω (Ohms) for the same levels (L1 and L2) of metallization. Differences in these values may be from several factors, including the partial filling of microconduits 34, in this example, low Galinstan 42 wettability, electrical mismatch between the example Galinstan 42 and the example silver (Ag) as the silver-ink cap 62 and the resulting layers, uneven spreading of Galinstan in the inner surfaces of the microconduits as shown in the partially filled microchannel of FIG. 2F, and the bimetallic junction depth effect as shown in FIG. 2G.

The maximum-minimum height difference of the 2.5D/3D microelectrodes 44 was about 300 μm. Average surface roughness of the electrodes 44 was obtained to be about 56 μm as shown generally at FIGS. 3B and 3C. This microelectrode 44 profile provides a 2.5D/3D configuration after the example silver-paste capping 62 suitable for interrogation and stimulation of electrogenic cell constructs.

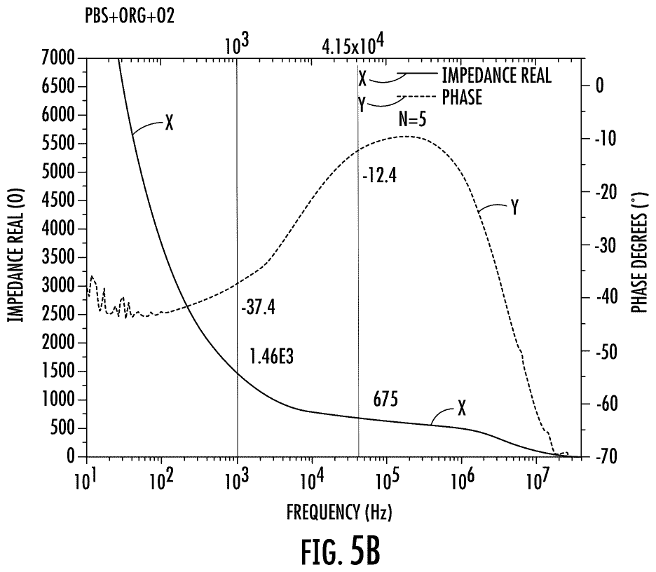

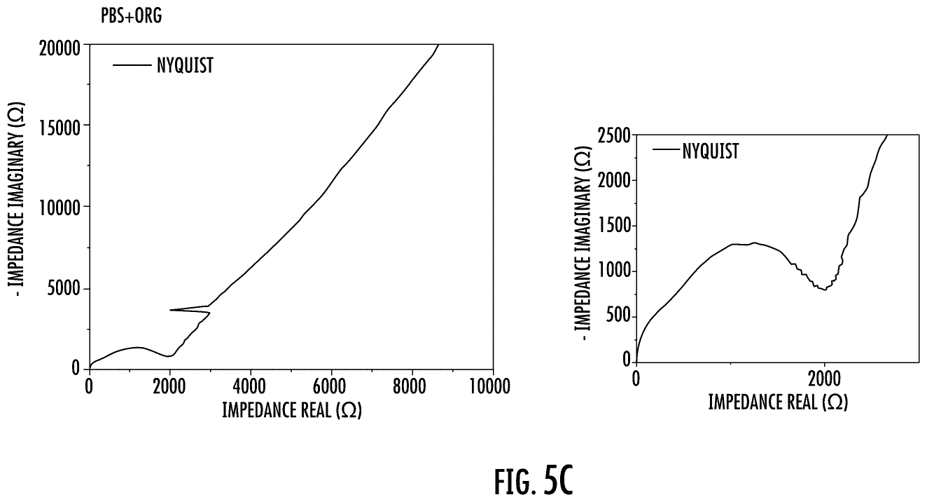

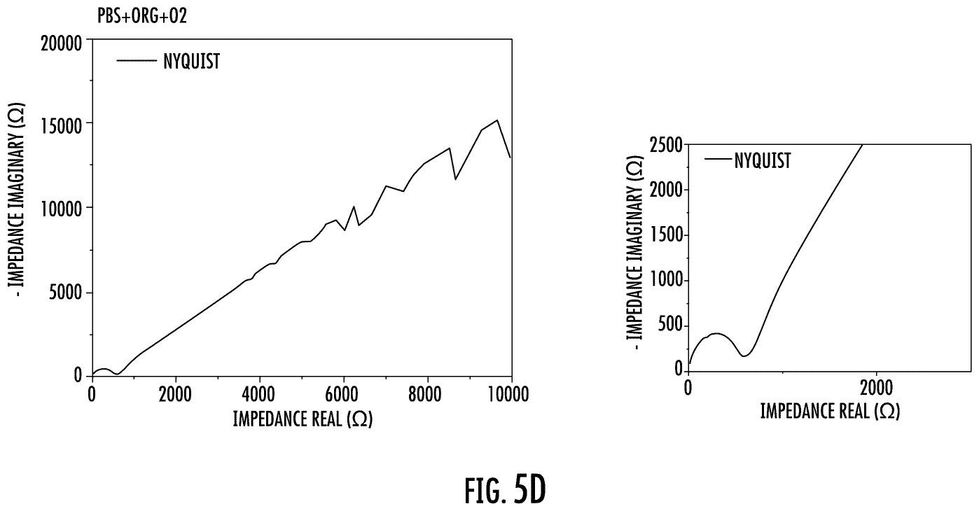

The bode plots are shown in FIGS. 5A and 5B for Organoids+PBS (FIG. 5A) and Organoids+PBS+O₂ (FIG. 5B), which show a decrease in Real Impedance at 1 KHz from 3.9 kΩ/−45° to 1.4 kΩ/−37°, suggesting a lower barrier integrity upon gas injection due to reduction in tight junctions of the organoids' samples for O₂ perfusion. Organoids 80 coverage on the array of microelectrodes 44 is determined by comparing Real Impedance at 41.5 kHz, which was also reduced upon O₂ injection from 2 kΩ/18° to 0.6 kΩ/12°. Generally, the PBS plus synthetic organoids had barrier integrity 3.9 kΩ at 1 KHz and MBA coverage 2 kΩ at 41.5 kHz and PBS plus the synthetic organoids plus O₂ had barrier integrity 1.4 kΩ at 1 KHz and MEA coverage 0.1 kΩ at 41.5 kHz.

On the other hand, the Nyquist plots shown in FIGS. 5C and 5D show the diffusion response at low frequencies of the microelectrodes 44 with the calculated surface roughness of ˜300 μm, which is typical for biomedical applications when polarizable electrodes are part of the device. The overall graph of the Nyquist plot is shown in the left side, which the enlarged section of the right shows the drop in imaginary impedance. At high frequencies, an evident shifting in the kinetic region from about 2.5 kΩ to about 0.5 kΩ is shown in the Real Impedance horizontal axis. This is given by the position and size of the distorted semicircle at the right-side extension, and its intersection with the ordinates when comparing both loci. A decay from 1.5 kΩ to 0.5 kΩ in the Imaginary Impedance value is observed on the semicircle peak (time-constant related) at a condition of abundance of O₂ (FIGS. 5C and 5D smaller insets at the right hand side).

Rapid prototyping with 3D printing technology along with the use of exotic materials allowed the co-development of a package/device 20 aimed to study organoids 80. The increase in the electrode 44 density may be achieved by shrinking the diameter of the microconduits 34 and/or adding more levels to the 3D printed platform. Limitation of spatial resolution on the MEA device 20 may be imposed by the limits on the 3D printing technology.

Electrochemical Impedance Spectroscopic (EIS) results evaluated at 1 KHz from Bode plots, suggest a lower barrier integrity upon gas injection due to reduction in tight junctions of the synthetic organoids 80 samples caused by O₂ perfusion. Organoids 80 coverage was also evaluated at 41.5 kHz depicting a reduction in its value from addition of O₂ gas to the microchamber 30. In both cases, the impedance reduction was about three-fold from its reference value before O₂ injection, also demonstrated by the reduction in the charge transfer resistance in the kinetic region of the Nyquist plot.

Further investigation in the electrical resistance measurements for the metallized microconduits 34 may be accomplished. Equivalent electrical circuit modeling, finite element modeling of microchannels liquid metal flow, bimetallic interface analysis, material compatibility, hydrophilicity, and hydrophobicity tests may also be conducted.

These results demonstrate that rapid microfabrication technologies and novel materials open new avenues for the development of a new generation of microfluidic platforms that form the MEA device 20 and integrates multi-phase inputs, 2.5D/3D microelectrodes and multi-modal control of reaction microchambers.

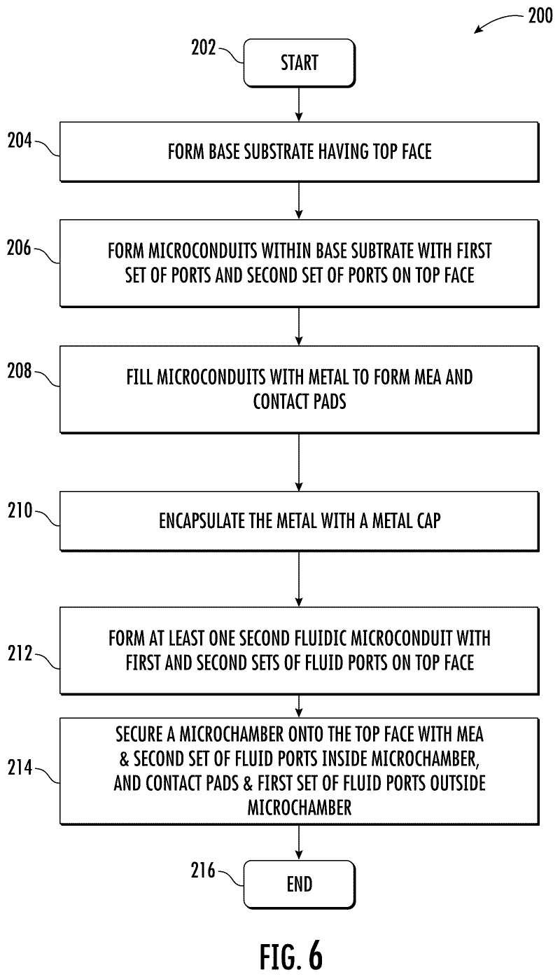

Referring now to FIG. 6, there is illustrated generally at 200 a high-level flowchart of a method of making the 3D MEA device 20 in accordance with a non-limiting example. The process starts (Block 202) and the base substrate 24 having a top face 28 is formed (Block 204) such as by 3D printing as described above. A first plurality of microconduits 34 is formed within the base substrate 24 with the first set of ports 38 and second set of ports 40 on the top face 28 and spaced in those areas after the microchamber 30 is secured onto the top face (Block 206). The microconduits 34 are filled with metal 42, such as the described liquid metal, to form the MEA 44 and contact pads 48 (Block 208). The metal 42 is encapsulated with a metal cap 62 (Block 210). At least one second fluidic microconduit 52 is formed with at least first and second sets of fluid ports 56,58 on the top face 28 (Block 212). At least one microchamber 30 is secured onto the top face 28 with the MEA 44 and second set of fluid ports 58 inside the microchamber and contact pads 48 and first set of fluid ports 56 outside the microchamber (Block 214). The process ends (Block 216).

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

1. A three-dimensional (3D), microchannel-based, microelectrode array (MEA) device for electrophysiological studies, comprising:
a base substrate having a top face;
at least one microchamber secured onto the top face;
a first plurality of microconduits formed within the base substrate, each of the first plurality of microconduits comprising a first set of ports on the top face outside the at least one microchamber and a second set of ports on the top face within the at least one microchamber, and a metal filling the microconduits to form a microelectrode array (MEA) on the top face within the at least one microchamber and contact pads on the top face outside the at least one microchamber; and
at least one second microconduit comprising a first set of fluid ports on the top face outside the at least one microchamber and a second set of fluid ports on the top face within the microchamber.

2. The 3D MEA device of claim 1 wherein said base substrate comprises a 3D printed substrate.

3. The 3D MEA device of claim 1 wherein said metal comprises a liquid metal, including at least one of Galinstan, Gallium, Eutectic Gallium-Indium (EGaIn), and Mercury.

4. The 3D MEA device of claim 3 wherein said MEA and contact pads each comprise a metallic cap encapsulating the liquid metal within the first plurality of microconduits.

5. The 3D MEA device of claim 1 wherein said microchamber includes at least one set of an output and input microfluidic ports.

6. The 3D MEA device of claim 1 comprising an organoid within the at least one microchamber on the top face at the MEA.

7. The 3D MEA device of claim 1 wherein the microelectrode array comprises microelectrodes having a maximum-minimum height difference about 300 micrometers.

8. A three-dimensional (3D), microchannel-based, microelectrode array (MEA) device for electrophysiological studies, comprising:
a base substrate having a top face;
at least one microchamber secured onto the top face;
a first plurality of microconduits formed within the base substrate, each of the first plurality of microconduits comprising a first set of ports on the top face outside the at least one microchamber and a second set of ports on the top face within the at least one microchamber, and a metal filling the microconduits to form a microelectrode array (MEA) on the top face within the at least one microchamber and contact pads on the top face outside the at least one microchamber;
a second plurality of microconduits comprising a first set of fluid ports on the top face outside the at least one microchamber and a second set of fluid ports on the top face within the at least one microchamber; and
organoids within the microchamber on the top face of the MEA, said organoids comprising at least one of biological, synthetic, 3D biological entity, regular, and 3D biological construct.

9. The 3D MEA device of claim 8 wherein said base substrate comprises a 3D printed substrate.

10. The 3D MEA device of claim 8 wherein said metal comprises a liquid metal, including at least one of Galinstan, Gallium, Eutectic Gallium-Indium (EGaIn), and Mercury.

11. The 3D MEA device of claim 10 wherein said MEA and contact pads each comprise a metallic cap encapsulating the liquid metal within the first plurality of microconduits.

12. The 3D MEA device of claim 8 wherein said at least one microchamber includes at least one set of output and input microfluidic ports.

13. The 3D MEA device of claim 8 wherein the microelectrode array comprises microelectrodes having a maximum-minimum height difference about 300 micrometers.

14. A method of forming a three-dimensional (3D), microchannel-based, microelectrode array (MEA) device for electrophysiological studies, comprising:
forming a base substrate having a top face;
forming a first plurality of microconduits within the base substrate, each of the first plurality of microconduits comprising a first set of ports on the top face and a second set of ports on the top face;
filling the microconduits with a metal to form a microelectrode array (MEA) on the top face and contact pads on the top face;
metal capping the filled microconduits;
forming at least one second microconduit comprising a first set of fluid ports on the top face and a second set of fluid ports on the top face; and
securing at least one microchamber onto the top face with the MEA inside the at least one microchamber and contact pads outside the at least one microchamber, and a first set of fluid ports outside the at least one microchamber and second set of fluid ports within the at least one microchamber.

15. The method of claim 14 comprising 3D printing the base substrate.

16. The method of claim 14 comprising filling the microconduits with a liquid metal comprising at least one of Galinstan, Gallium, Eutectic Gallium-Indium (EGaIn), and Mercury.

17. The method of claim 14 wherein said microchamber includes at least one set of output and input microfluidic ports.

18. The method of claim 14 comprising forming organoids within the at least one microchamber on the top face at the MEA.

19. The method of claim 14 wherein the microelectrode array comprises microelectrodes having a maximum-minimum height difference about 300 micrometers.
